(19) United States
(12) Patent Application Publication
(10) Pub. No.: US 2009/0253984 A1
(43) Pub. Date: Oct. 8, 2009

(54) MEDICAL IMAGING METHOD AND MEDICAL IMAGING SYSTEM

(75) Inventors: Masao YUI, Otawara-shi (JP); Junichi Makita, Tustin, CA (US); Yoshio Machida, Nasushiobara-shi (JP)

Correspondence Address: NIXON & VANDERHYE, PC, 901 NORTH GLEBE ROAD, 11TH FLOOR, ARLINGTON, VA, 22203, US

(73) Assignees: KABUSHIKI KAISHA TOSHIBA, Tokyo (JP); TOSHIBA MEDICAL SYSTEMS CORPORATION, Tochigi (JP)

(21) Appl. No.: 12/412,897

(22) Filed: Mar. 27, 2009

(30) Foreign Application Priority Data
Apr. 2, 2008 (JP) 2008-096312

(51) Int. Cl. A61B 5/055

(52) U.S. Cl. 600/420

(57) ABSTRACT

In a medical imaging method of taking a tomographic image of a sample into which a contrast medium is injected by the use of a medical imaging system, a monitoring operation is performed on the sample into which the contrast medium is injected and then an imaging scan for reconstructing the tomographic image is performed. The monitoring operation is to repeat the steps of: (1) performing a monitoring scan for collecting data representing a concentration distribution of the contrast medium in a monitoring region of the sample, (2) reconstructing a reconstructed image representing the concentration distribution of the contrast medium in the monitoring region from the data acquired by the monitoring scan, (3) calculating a maximum signal value from a reconstructed image newly reconstructed and the reconstructed image previously reconstructed every pixel and generating a monitoring image including the maximum signal value calculated every pixel whenever the reconstructed image is newly reconstructed, and (4) monitoring whether a start time of the imaging scan comes in, until the start time comes in.

### CROSS-REFERENCE TO RELATED APPLICATIONS

This application is based upon and claims the benefit of priority from prior Japanese Patent Application No. 2008-096312, filed Apr. 2, 2008, the entire contents of which are incorporated herein by reference.

### BACKGROUND OF THE INVENTION

1. Field of the Invention

The present invention relates to a medical imaging method and a medical imaging system for imaging a fluid such as blood.

2. Description of the Related Art

Magnetic resonance angiography (MRA) is an example of applications of a magnetic resonance imaging (MRI) method. The MRI can implement the MRA using various contrast generating principles such as a TOF (Time-Of-Flight) effect or a phase shift effect. A method of rapidly injecting a contrast medium having a longitudinal relaxation time (T1) reducing effect into a human body and imaging the blood into which the contrast medium is mixed is most widely used (hereinafter, referred to as “contrast MRA”). Images of the blood vascular system all over the body such as the neck, the head, and the foot in addition the main artery and the renal artery can be obtained by the contrast MRA.

The imaging time in the contrast MRA is usually in the range of several seconds to several tens seconds. Accordingly, one contrast medium injection permits only one imaging.

On the other hand, since the contrast medium is usually injected from the outside of a region of interest, the time point when the contrast medium arrives at the region of interest to obtain an image having excellent contrast is later than the contrast medium injection time. In addition, the delay time depends on the heart rate, the blood pressure, and the blood flow rate of a sample and thus is not constant.

In view of these points, it is important in the contrast MRA to properly set the imaging time point and studies thereof were made in the past as follows.

For example, as a first technique, a technique of continuously acquiring magnetic resonance signals from only a defined monitoring region (the inside of the main artery upstream of the region of interest) close to the region of interest before the contrast MRA imaging, providing a temporal variation in signal strength thereof to an operator, and starting the imaging in synchronization with the time point when the signal strength increases equal to or greater than a threshold value is known (PCT Japanese Translation Patent Publication No. 2000-511789).

A second technique was suggested as a substitute of the first technique, and monitors a relative wide range using the fluoroscopy employing a two-dimensional imaging method and directly provides an arrival of the contrast medium at the region of interest as a variation of an image signal (see Radiology, vol. 205, p 137 (1997)).

In the second technique, since a wide range can be continuously observed, a state where the contrast medium injected from the brachium of a sample is passing through the lungs, the atriums, the ventricles, or the main artery is displayed in real time. An operator estimates the time of the arrival of the contrast medium at the region of interest on the basis of the display and instructs to start the contrast MRA imaging.

As a third technique, a technique of performing a subtraction process or a multi-slice-data maximum value process on the image signals obtained in the second technique to clearly display the state where the contrast medium arrives at the region of interest was known (see JP-A-2003-235827).

However, in the first technique, the increase in signal strength could not be caught satisfactorily as described in Radiology, vol. 203, p 275 (1997). It is considered that this is because blood flow signals could not be satisfactorily measured due to the breathing or the bodily movement in the monitoring region. The first technique also has a problem that it is necessary to properly position the small-volume monitoring region and the operations are complex.

On the other hand, in the second and third techniques, it is difficult for an operator having a poor experience in contrast MRA examination to accurately catch the arrival of the contrast medium at the region of interest and thus might not start the contrast MRA imaging at a proper time. The reason is that signals of the monitoring image vary due to the heartbeat or a patient's movement and a variation in signal due to the arrival of the contrast medium is overlapped on the variation. It is difficult for an operator having a poor experience in contrast MRA examination to distinguish the variation in signal due to the contrast medium from the variation in signal due to the heartbeat. Accordingly, the operator may erroneously determine that the variation in signal in the region of interest due to the heartbeat results from the arrival of the contrast medium at the region of interest and may perform the imaging too early, thereby acquiring an MRA image having poor expression.

### BRIEF SUMMARY OF THE INVENTION

In view of the above-mentioned problems, there is a need for an image capable of reducing the frequency of erroneously considering a variation in signal due to the heartbeat or a patient's movement as a variation in signal due to the arrival of a contrast medium.

According to a first aspect of the invention, there is provided a medical imaging method of taking a tomographic image of a sample into which a contrast medium is injected by the use of a medical imaging system, wherein a monitoring operation is performed on the sample into which the contrast medium is injected and then an imaging scan for reconstructing the tomographic image is performed and wherein the monitoring operation is to repeat the steps of: (1) performing a monitoring scan for collecting data representing a concentration distribution of the contrast medium in a monitoring region of the sample; (2) reconstructing a reconstructed image representing the concentration distribution of the contrast medium in the monitoring region from the data acquired by the monitoring scan; (3) calculating a maximum signal value from a reconstructed image newly reconstructed and the reconstructed image previously reconstructed every pixel and generating a monitoring image including the maximum signal value calculated every pixel whenever the reconstructed image is newly reconstructed; and (4) monitoring whether a start time of the imaging scan comes in, until the start time comes in.

According to a second aspect of the invention, there is provided a medical imaging system for taking a tomographic image of a region of interest in a sample into which a contrast medium is injected, including: a collection unit collecting data representing a concentration distribution of the contrast medium; a first control unit controlling the collection unit to collect the data on a monitoring region of the sample; a first reconstruction unit reconstructing a reconstructed image representing the concentration distribution of the contrast medium in the monitoring region from the data collected by the collection unit under the control of the first control unit; a maximum-value image generating unit calculating a maximum signal value from the reconstructed image newly reconstructed and the reconstructed image previously reconstructed by the first reconstruction unit every pixel and generating a maximum-value image including the maximum signal value calculated every pixel whenever the reconstructed image is newly reconstructed by the first reconstruction unit; a monitoring unit monitoring whether a start time of an imaging scan comes in; a second control unit controlling the first control unit, the first reconstruction unit, and the maximum-value image generating unit to repeat (1) the collection of the data on the monitoring region, (2) the reconstruction of the reconstructed image of the monitoring region, and (3) the generation of the maximum-value image until the monitoring unit determines that the start time comes in; a third control unit controlling the collection unit to collect the data on the region of interest after the start time comes in; and a second reconstruction unit reconstructing a tomographic image of the region of interest from the data collected by the collection unit under the control of the third control unit.

According to a third aspect of the invention, there is provided a medical imaging system for taking a magnetic resonance image representing a configuration of a flow channel of a fluid in a sample, including: a collection unit repeatedly collecting magnetic resonance data from a region of interest including the flow channel of the fluid a plurality of times while changing a predetermined parameter influencing an imaging condition of the fluid; a reconstruction unit reconstructing a plurality of reconstructed images of the region of interest from the magnetic resonance data collected the plurality of times by the collection unit; and a maximum-value image generating unit calculating a maximum signal value from the reconstructed image newly reconstructed and the reconstructed image previously reconstructed by the reconstruction unit every pixel and generating a maximum-value image including the maximum signal value calculated every pixel whenever the reconstructed image is newly reconstructed by the reconstruction unit.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram schematically illustrating a configuration of a magnetic resonance imaging (MRI) machine according to an embodiment of the invention.

FIG. 2 is a flowchart illustrating a flow of a contrast MRA imaging process according to a first embodiment of the invention, which is performed by a host computer shown in FIG. 1.

FIG. 3 is a diagram illustrating a variation in signal strength of a pixel in a displaying image according to the first embodiment of the invention.

FIG. 4 is a diagram illustrating an image displayed to monitor a contrast medium according to a related art.

FIG. 5 is a diagram illustrating a displaying image generated according to the first embodiment of the invention.

FIG. 6 is a flowchart illustrating a flow of a contrast MRA imaging process according to a second embodiment of the invention, which is performed by the host computer shown in FIG. 1.

FIG. 7 is a diagram illustrating a variation in signal strength of a pixel in a displaying image according to the second embodiment of the invention.

FIG. 8 is a flowchart illustrating a flow of the contrast MRA imaging process according to a third embodiment of the invention, which is performed by the host computer shown in FIG. 1.

### DETAILED DESCRIPTION OF THE INVENTION

Hereinafter, preferred embodiments of the invention will be described with reference to the accompanying drawings.

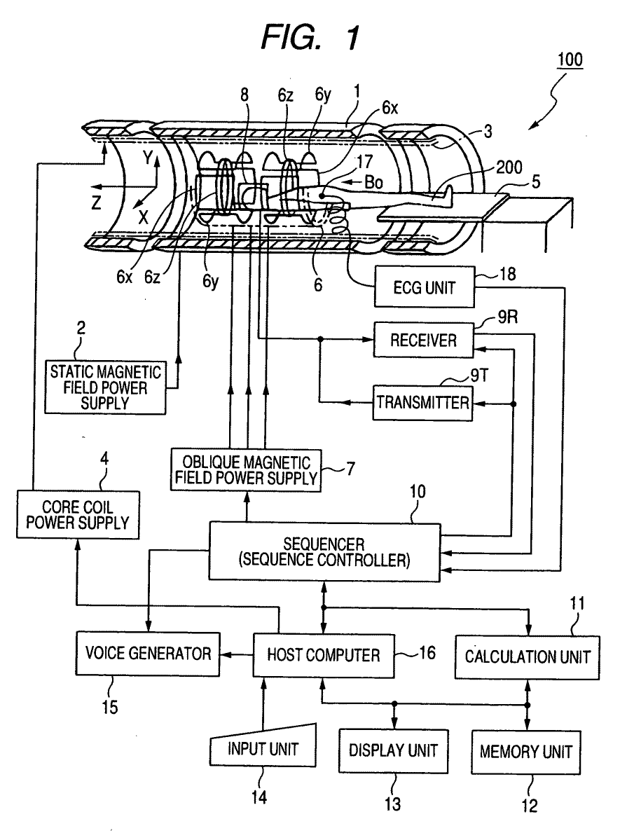

FIG. 1 is a diagram schematically illustrating a magnetic resonance imaging (MRI) machine 100 according to an embodiment of the invention.

The MRI machine 100 includes a bed section, a static magnetic field generator, an oblique magnetic field generator, a transceiver unit, and a control and calculation section. The MRI machine 100 includes as constituent elements of the units a magnet 1, a static magnetic field power supply 2, a core coil 3, a core coil power supply 4, a top plate 5, an oblique magnetic field coil unit 6, an oblique magnetic field power supply 7, an RF coil unit 8, a transmitter 9T, a receiver 9R, a sequencer (sequence controller) 10, a calculation unit 11, a memory unit 12, a display 13, an input unit 14, a voice generator 15, and a host computer 16. An ECG measuring unit measuring an ECG signal as a signal representing a cardiac time phase of a sample 200 is connected to the MRI machine 100.

The static magnetic field generator includes the magnet 1 and the static magnetic field power supply 2. For example, a superconductor magnet or a normal conductor magnet can be used as the magnet 1. The static magnetic field power supply 2 supplies current to the magnet 1. The static magnetic field generator generates a static magnetic field B₀ in a cylindrical space (diagnosis space) to which the sample 200 is loaded. The magnetic field direction of static magnetic field B₀ is substantially parallel to an axis direction (Z axis direction) of the diagnosis space. The static magnetic field generator further includes the core coil 3. The core coil 3 generates a correcting magnetic field uniformizing the static magnetic field with a supply of current from the core coil power supply 4 under the control of the host computer 16.

The bed section serves to load the top plate 5 on which the sample 200 is placed to the diagnosis space or to unload the top plate from the diagnosis space.

The oblique magnetic field generator includes the oblique magnetic field coil unit 6 and the oblique magnetic field power supply 7. The oblique magnetic field coil unit 6 is disposed inside the magnet 1. The oblique magnetic field coil unit 6 includes three coils 6x, 6y, and 6z generating the oblique magnetic fields in an X axis direction, a Y axis direction, and a Z axis direction perpendicular to each other. The oblique magnetic field power supply 7 supplies pulse current for generating the oblique magnetic fields to the coil 6x, the coil 6y, and the coil 6z under the control of the sequencer 10. By controlling the pulse current supplied from the oblique magnetic field power supply 7 to the coils 6x, 6y, and 6z, the oblique magnetic field generator synthesizes the oblique magnetic fields in three axes (X axis, Y axis, and Z axis) directions which are physical axes and arbitrarily sets the oblique magnetic fields in logical axes directions including an oblique magnetic field G_{S} in slice direction, an oblique magnetic field G_{E} in a phase encoding direction, and an oblique magnetic field G_{R} in a reading direction (frequency encoding direction) perpendicular to each other. The oblique magnetic fields G_{S}, G_{E}, and G_{R} in the slice direction, the phase encoding direction, and the reading direction overlaps with the static magnetic field B₀.

The transceiver unit includes the RF coil unit 8, the transmitter 9T, and the receiver 9R. The RF coil unit 8 is disposed in the vicinity of the sample 200 in the diagnosis space. The transmitter 9T and the receiver 9R are connected to the RF coil unit 8. The transmitter 9T and the receiver 9R operate under the control of the sequencer 10. The transmitter 9T supplies the RF coil unit 8 with an RF current pulse of a Larmor frequency for generating nuclear magnetic resonance (NMR). The receiver 9R receives an MR signal (high-frequency signal) such as an echo signal received by the RF coil unit 8, performs various signal processing such as pre-amplification, intermediate frequency transformation, phase detection, low-frequency amplification, and filtering on the input MR signal, converts the processed signal in an analog-to-digital conversion manner, and generates digital data (raw data).

Operations of several embodiments of the MRI machine 100 having the above-mentioned configuration will be described now.

### First Embodiment

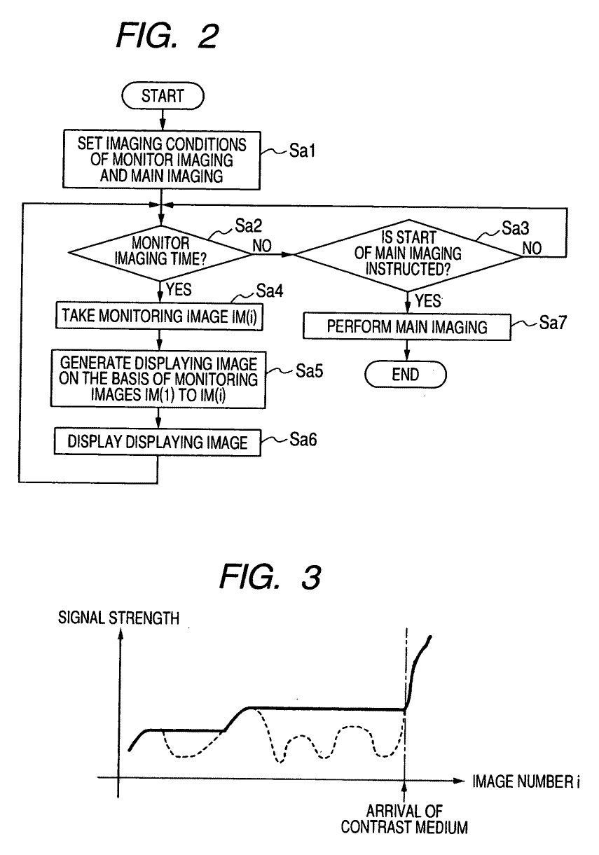

FIG. 2 is a flowchart illustrating a flow of a contrast MRA imaging process according to a first embodiment of the invention, which is performed by the host computer 16.

In Act Sa1, the host computer 16 sets imaging conditions of a monitor imaging and a main imaging on the basis of an operator's instruction or the like.

In Acts Sa2 and Sa3, the host computer 16 monitors whether the time of the monitor imaging comes in or waits for a start instruction of the main imaging from the operator.

Here, the monitor imaging is performed with a constant time interval until it is instructed to start the main imaging. When the time of each constant time interval comes in, the host computer 16 brings the flow from Act Sa2 to Act Sa4. In Act Sa4, the host computer 16 controls the units to perform the monitor imaging. Generally, a gradient and echo pulse sequence is used in the monitor imaging. In the monitor imaging, magnetic resonance signals of a region set as a monitoring target are collected and a monitoring image IM(i) is reconstructed on the basis of the magnetic resonance signals. Here, “i” represents an image number of the monitoring image, is “1” just after the flow shown in FIG. 2 is started, and increases by 1 whenever the monitor imaging is performed. The monitoring image IM(i) may be a single image or may include plural images.

In Act Sa5, the host computer 16 generates a displaying image on the basis of all the monitoring images IM(1) to IM(i) generated hitherto. Specifically, the host computer 16 calculates the maximum value of signal strength in the monitoring images IM(1) to IM(i) every pixel included in the monitoring image. The displaying image is generated from an image acquired by setting the maximum values as the signal strength of the pixels. That is, when the signal strength of a pixel P in a monitoring image IM(k) among the monitoring images IM(1) to IM(i) is SignalA[P,k], the signal strength SignalB[P,i] of the pixel P in the displaying image is calculated by Expression 1.

SignalB[P,i]=Maximum of SignalA[P,k] (k=1 , , , i) (1)

Expression 1 can be rewritten as Expression 2.

SignalB[P,i]=Maximum(SignalA[P,i], SignalB[P,i−1]) (2)

The host computer 16 may calculate the signal strength of the pixels in the displaying image using any one of Expressions 1 and 2. However, it is preferable that Expression 2 is used, because the processing load of the host computer 16 can be reduced.

In Act Sa6, the host computer 16 displays the displaying image on the display 13. At this time, when the displaying image was being displayed on the display 13, the display on the display 13 is updated to display the displaying image newly generated instead.

Then, the host computer 16 returns the flow to the wait state of Acts Sa2 and Sa3.

In this way, the monitoring image IM(i) is obtained with a constant time interval and the displaying image is updated, until it is instructed to start the main imaging.

FIG. 3 is a diagram illustrating a variation in signal strength of a pixel P in the displaying image according to the first embodiment of the invention.

In FIG. 3, the solid line represents the variation in signal strength of the pixel P in the displaying image and the broken line represents the variation in signal strength of the pixel P in the monitoring image IM(i). As can be seen from FIG. 3, the signal strength of the pixel P in the monitoring image IM(i) varies due to the influence of the heartbeat of the sample 200 or the like before the contrast medium arrives at a position corresponding to the pixel P. However, since the signal strength of the pixel P in the displaying image has the maximum value of the signal strength of the pixel P in the monitoring image IM(i), the variation occurs due to the influence of the heartbeat of the sample 200 at the first but the signal strength gets substantially constant with the lapse of a certain time. When the contrast medium reaches the position corresponding to the pixel P, the signal strength of the pixel P in the monitoring image IM(i) rapidly increases and thus the variation appears in the signal strength in the displaying image.

Therefore, by starting the monitor imaging from a certain time before the contrast medium reaches an imaging range in the monitor image IM(i), a certain period of time (which can be referred to as “training period”) is secured until the signal strength in the monitoring image IM(i) varies due to the influence of the contrast medium. Then, the signal variation due to the contrast medium clearly appears in the displaying image from the state the signal variation due to the heartbeat or the like disappears therefrom. Accordingly, the operator can easily confirm that the contrast medium reaches the corresponding position by observing the displaying image displayed on the display 13 and checking the rapid variation in signal strength. The training period is a period of time when at least several heartbeats or breathing are made and it is considered that several seconds to several tens seconds are proper as the training period in actuality.

In this embodiment, the signal strength of the pixel corresponding to a position where the contrast medium once arrives is not reduced even when the contrast medium passes through the position again. Accordingly, the displaying image is changed so that the region through which the contrast medium passes is expressed in a high signal state. Therefore, the operator can easily grasp a state where the contrast medium is flowing through the body of the sample 200 on the basis of the displaying image and can also estimate the time point when the contrast medium reaches the region of interest which is a target of the main imaging.

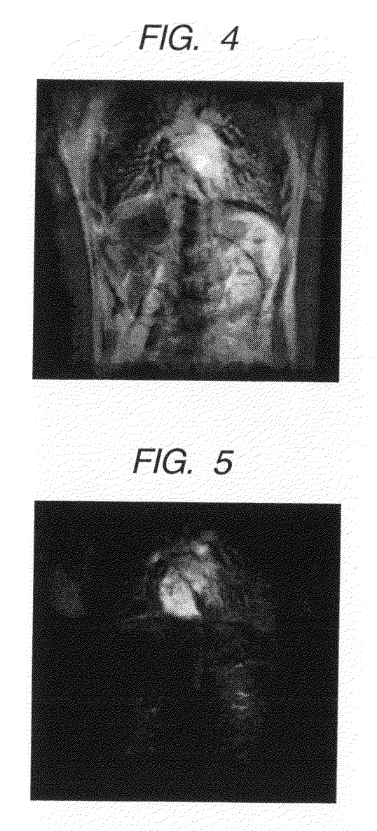

FIG. 4 is a diagram illustrating an image displayed to monitor the contrast medium according to a related art. FIG. 5 is a diagram illustrating an example of the displaying image. The images shown in FIGS. 4 and 5 are acquired substantially at the time point when the contrast medium reaches the abdominal main artery from the left ventricle and the thoracic main artery. In the image shown in FIG. 4, the increase in signal due to the contrast medium, the unevenness in signal strength due to the heartbeat, the artifacts, and the like are mixed and thus it is difficult to observe only the contrast medium. On the contrary, in the image shown in FIG. 5, the regions (heart, main artery, and lung blood vessels) which the contrast medium reaches until the image is acquired are expressed and unnecessary signals due to the heartbeat or the artifacts are small. In the first embodiment, the image shown in FIG. 5 is continuously displayed and thus the moving state of the contrast medium can be clearly observed therefrom.

The operator determines the start time of the main imaging on the basis of the observation of the displaying image and instructs to start the main imaging. In response to this instruction, the host computer 16 brings the flow from Act Sa3 to Act Sa7. In Act Sa7, the host computer 16 controls the units to perform the main imaging of the contrast MRA. The operations of the unit for the main imaging may be known operations. When the main imaging is ended, the host computer 16 ends the flow.

### Second Embodiment

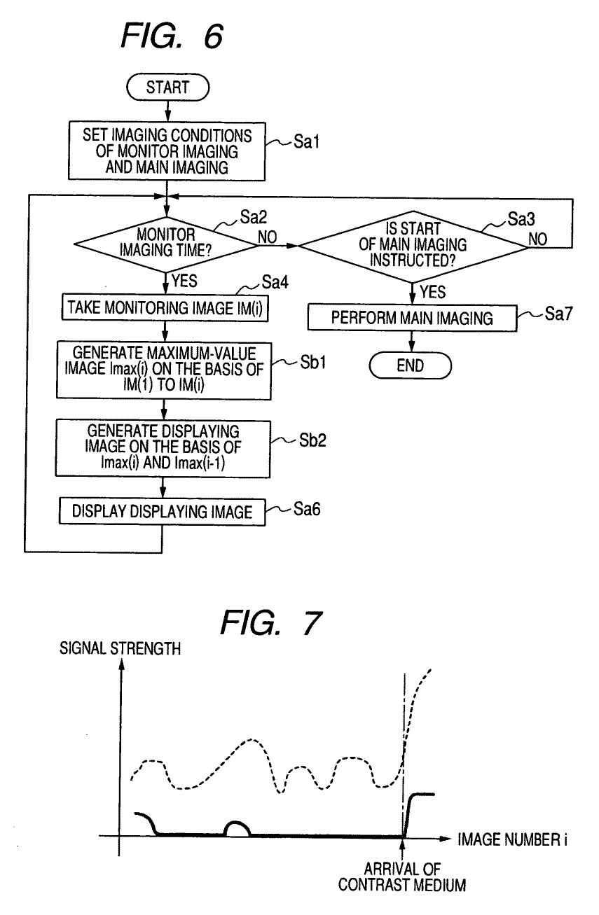

FIG. 6 is a flowchart illustrating a flow of a contrast MRA imaging process according to a second embodiment of the invention, which is performed by the host computer 16. Acts of performing the same processes as shown in FIG. 2 are referenced by like reference numerals or signs and detailed description thereof is omitted.

The second embodiment is different from the first embodiment, in the method of calculating the signal strength of each pixel for generating the displaying image. In the second embodiment, the host computer 16 performs the processes of Acts Sb1 and Sb2 instead of Act Sa5 in the first embodiment, as shown in FIG. 6.

In Act Sb1, the host computer 16 generates an image (hereinafter, referred to as “maximum-value image”) Imax(i) in which each pixel is expressed by a signal strength SignalB[P,i], similarly to the displaying image in the first embodiment.

In Act Sb2, the host computer 16 generates the displaying image on the basis of the maximum-value image Imax(i) newly generated and the maximum-value image Imax(i−1) generated just before. Specifically, the host computer 16 calculates a difference between the signal strength in the maximum-value image Imax(i) and the signal strength of the maximum-value image Imax(i−1) every pixel. An image obtained by setting the differences as the signal strength of the corresponding pixels is generated as the displaying image. That is, the signal strength SignalC[P,i] of a pixel P in the displaying image is calculated by Expression 3 using SignalB[P,i] obtained from Expression 1 or 2.

SignalC[P,i]=SignalB[P,i]−SignalB[P,i−1] (3),

FIG. 7 is a diagram illustrating a variation in signal strength of a pixel P in the displaying image according to the second embodiment of the invention.

In FIG. 7, the solid line represents the variation in signal strength of the pixel P in the displaying image and the broken line represents the variation in signal strength of the pixel P in the monitoring image IM(i). As can be seen FIG. 7, the variation due to the influence of the heartbeat or the like of the sample 200 rarely appears in the signal strength of the pixel P in the displaying image, but the signal strength due to the heartbeat is almost zero in the most periods. When the contrast medium arrives at the position corresponding to the pixel P, SignalB[P,i] rapidly increases as described above and thus the variation also appears in the signal strength of the displaying image.

Therefore, when a certain training period is secured similarly to the first embodiment, the signal variation due to the contrast medium clearly appears in the displaying image from the state where the signal variation due to the heartbeat or the like disappears. Accordingly, the operator can easily confirm that the contrast medium arrives at the corresponding position by observing the displaying image displayed on the display 13 and checking the rapid variation in signal strength.

### Third Embodiment

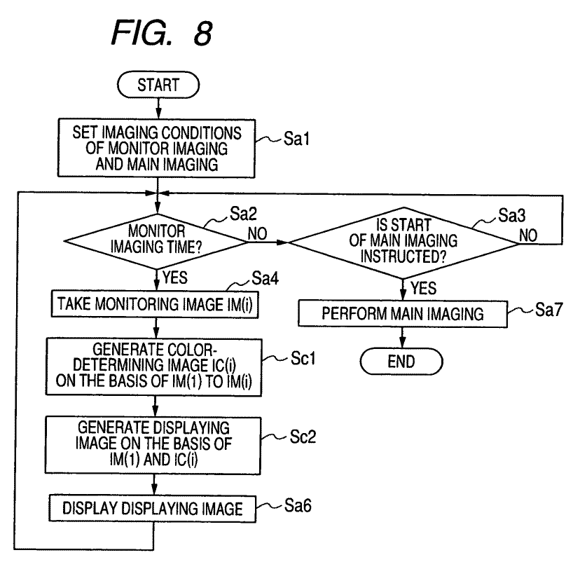

FIG. 8 is a flowchart illustrating a flow of a contrast MRA imaging process according to a third embodiment of the invention, which is performed by the host computer 16. Acts of performing the same processes as FIG. 2 are referenced by like reference numerals or signs and detailed description thereof is omitted.

The third embodiment is different from the first embodiment, in that a display characteristic of the pixels in the displaying image is changed. In the third embodiment, the host computer 16 performs the processes of Acts Sc1 and Sc2 instead of Act Sa5 in the first embodiment, as shown in FIG. 8.

In Act Sc1, the host computer 16 generates a color-determining image IC(i) from the image in which the pixels are expressed by the signal strength SignalB[P,i] similarly to the displaying image in the first embodiment or the image in which the pixels are expressed by the signal strength SignalC[P,i] similarly to the displaying image in the second embodiment.

In Act Sc2, the host computer 16 generates the displaying image on the basis of the monitoring image IM(i) and the color-determining image IC(i). Specifically, the host computer 16 determines whether a display color should be changed every pixel, depending on whether the signal strength of the color-determining image IC(i) is greater than a predetermined threshold value. The host computer 16 generates the displaying image by changing the display color of only the pixels in the monitoring image IM(i) of which the display color is determined as being changed. For example, when the original display colors of the monitoring images IM(i) are in a gray scale, the display color of the pixel of which the display color is determined as being changed is changed to a color scale. At this time, the signal strength of the pixels employ those of the monitoring image IM(i).

According to the third embodiment, the operator can determine a place at which the contrast medium arrives and a place at which the contrast medium does not arrive on the basis of the display colors of the displaying image. The operator can easily confirm the moving state of the contrast medium by observing the temporal variation of a colored region or the movement and enlargement of the region.

In addition, according to the third embodiment, since the instantaneous heartbeat or the like is displayed along with the movement of the contrast medium, the operator can also confirm it.

This embodiment can be modified in various forms as follows.

The invention can be applied to an MRA imaging not using the contrast medium or an imaging using a fluid other than the blood as a target. A method of labeling the blood or the like to cause a change in contrast similar to the contrast medium such as an ASL (Arterial Spin Labeling) method is known for the MRA imaging not using the contrast medium. Alternatively, a method of changing the initial value of the signal strength by the use of a control pulse associated with the image contrast such as an MTC (Magnetization Transfer Contrast) pulse or an inversion pulse and observing the temporal variation thereof is known.

The ASL method is a technique of applying a pre-pulse for labeling the blood flow or the like and taking an image thereof. As such a method, a method of acquiring an image showing a state where the fluid such as the blood is flowing by taking plural images while changing the time from the application of the labeling pulse to the start of the data collection is known. That is, the ASL method is a technique of acquiring an image showing the changing state of the fluid such as the blood by repeating the imaging at plural time phases while changing a parameter. In the ASL method, it is general that a signal variation is analyzed and then various parameter images are generated and provided. The image displaying method included in the invention is useful for directly observing images at plural time phases in the ASL method. For example, by overlapping an image resulting from the combination of the displaying method of the invention and the ASL method with a diffusion-emphasized image, it is possible to provide information associated with a so-called diffusion-perfusion mismatch.

As the techniques of acquiring the plural time-phase images, an FBI (Flesh Blood Imaging) method and a flow-spoiled FBI method are known in addition. The FBI method is a technique of acquiring a T2 emphasized image, in which a T2 magnetization component of a fluid such as blood or infusion is emphasized, in synchronous imaging using an ECG signal or a PPG signal. In the FBI method, echo data is repeatedly collected every plural heartbeats with the delay of a predetermined time from a trigger signal synchronized with a reference wave such as an R wave representing a cardiac time phase of a sample. In the three-dimensional imaging using the FBI method, the echo data (volume data) corresponding to a predetermined slice encoded volume is collected every plural heartbeats.

The flow-spoiled FBI method is a technique of applying a spoiler oblique magnetic field pulse for controlling an artery signal at a systolic phase in addition to the FBI method. The parameter to be changed is a time from the generation of the reference wave such as an R wave representing the cardiac time phase of a sample to the start of data collection for reconstructing an image in the FBI method, and is the strength of a diphase pulse in the flow-spoiled FBI method. In both methods, the optimal time point for the synchronous imaging is determined from plural images. Therefore, by applying the techniques according to the above-mentioned embodiments to the imaging methods, the variation in signal strength due to the change of a parameter can be expressed more clearly than the variation in signal strength due to the other reasons and thus the optimal time phase can be easily determined. In the flow-spoiled FBI method, by performing the collection plural times with the change in strength of the diphase pulse and checking the variation in signal strength, it is possible to easily determined the optimal strength of the diphase pulse.

In this way, the display image may be generated by the use of the techniques according to the above-mentioned embodiments on the basis of plural images acquired by various imaging techniques of repeatedly taking an image at plural time phases while changing the parameter, in addition to the FBI method or the flow-spoiled FBI method. In this case, the invention can be used to generate a medical diagnosis image in the main imaging as well as the monitor imaging.

The invention can be applied to the other type of machines such as an X-ray CT (Computed Tomography) performing an imaging operation using an imaging method other than the MRI method.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical imaging method of taking a tomographic image of a sample into which a contrast medium is injected by the use of a medical imaging system,
wherein a monitoring operation is performed on the sample into which the contrast medium is injected and then an imaging scan for reconstructing the tomographic image is performed, and
wherein the monitoring operation is to repeat the steps of:
(1) performing a monitoring scan for collecting data representing a concentration distribution of the contrast medium in a monitoring region of the sample;
(2) reconstructing a reconstructed image representing the concentration distribution of the contrast medium in the monitoring region from the data acquired by the monitoring scan;
(3) calculating a maximum signal value from a reconstructed image newly reconstructed and the reconstructed image previously reconstructed every pixel and generating a monitoring image including the maximum signal value calculated every pixel whenever the reconstructed image is newly reconstructed; and
(4) monitoring whether a start time of the imaging scan comes in,
until the start time comes in.

2. A medical imaging system for taking a tomographic image of a region of interest in a sample into which a contrast medium is injected, the system comprising:
a collection unit collecting data representing a concentration distribution of the contrast medium;
a first control unit controlling the collection unit to collect the data on a monitoring region of the sample;
a first reconstruction unit reconstructing a reconstructed image representing the concentration distribution of the contrast medium in the monitoring region from the data collected by the collection unit under the control of the first control unit;
a maximum-value image generating unit calculating a maximum signal value from the reconstructed image newly reconstructed and the reconstructed image previously reconstructed by the first reconstruction unit every pixel and generating a maximum-value image including the maximum signal value calculated every pixel whenever the reconstructed image is newly reconstructed by the first reconstruction unit;
a monitoring unit monitoring whether a start time of an imaging scan comes in;
a second control unit controlling the first control unit, the first reconstruction unit, and the maximum-value image generating unit to repeat (1) the collection of the data on the monitoring region, (2) the reconstruction of the reconstructed image of the monitoring region, and (3) the generation of the maximum-value image until the monitoring unit determines that the start time comes in;
a third control unit controlling the collection unit to collect the data on the region of interest after the start time comes in; and
a second reconstruction unit reconstructing a tomographic image of the region of interest from the data collected by the collection unit under the control of the third control unit.

3. The system according to claim 2, further comprising a display unit displaying the maximum-value image generated by the maximum-value image generating unit,
wherein the monitoring unit waits for an operator's instruction to start the imaging scan based on the maximum-value image displayed on the display unit and determines whether the start time comes in on the basis of the time point when the start instruction is given.

4. The system according to claim 3, wherein the maximum-value image generating unit changes a display characteristic of the pixels in the maximum-value image depending on whether the maximum values of the pixels are equal to or greater than a threshold value.

5. The system according to claim 4, wherein the maximum-value image generating unit changes colors of the pixels in the maximum-value image as the display characteristic of the pixels.

6. The system according to claim 2, further comprising a graph generating unit generating a graph representing a temporal variation in signal value of some pixels in the maximum-value image generated by the maximum-value image generating unit,
wherein the monitoring unit determines whether the start time comes in on the basis of the graph generated by the graph generating unit.

7. The system according to claim 2, further comprising:
a differential image generating unit generating a differential image representing a differential value in signal value of each pixel between the maximum-value image newly generated by the maximum-value image generating unit and the latest maximum-value image previously generated by the maximum-value image generating unit; and
a display unit displaying the differential image generated by the differential image generating unit,
wherein the monitoring unit waits for an operator's instruction to start the imaging scan based on the differential image displayed on the display unit and determines whether the start time comes in on the basis of the time point when the start instruction is given.

8. The system according to claim 7, wherein the differential image generating unit changes a display characteristic of the pixels in the differential image depending on whether the differential values of the pixels are equal to or greater than a threshold value.

9. The system according to claim 8, wherein the differential image generating unit changes colors of the pixels in the differential image as the display characteristic of the pixels.

10. The system according to claim 2, further comprising:
a differential image generating unit generating a differential image representing a differential value in signal value of each pixel between the maximum-value image newly generated by the maximum-value image generating unit and the latest maximum-value image previously generated by the maximum-value image generating unit; and
a graph generating unit generating a graph representing a temporal variation in signal value of some pixels in the differential image generated by the differential image generating unit,
wherein the monitoring unit determines whether the start time comes in on the basis of the graph generated by the graph generating unit.

11. A medical imaging system for taking a magnetic resonance image representing a configuration of a flow channel of a fluid in a sample, the system comprising:
a collection unit repeatedly collecting magnetic resonance data from a region of interest including the flow channel of the fluid a plurality of times while changing a predetermined parameter influencing an imaging condition of the fluid;
a reconstruction unit reconstructing a plurality of reconstructed images of the region of interest from the magnetic resonance data collected the plurality of times by the collection unit; and
a maximum-value image generating unit calculating a maximum signal value from the reconstructed image newly reconstructed and the reconstructed image previously reconstructed by the reconstruction unit every pixel and generating a maximum-value image including the maximum signal value calculated every pixel whenever the reconstructed image is newly reconstructed by the reconstruction unit.

12. The system according to claim 11, wherein the parameter is a time phase in synchronous imaging.

13. The system according to claim 11, wherein the parameter is associated with an application of a control pulse influencing image contrast of the reconstructed image.

14. The system according to claim 11, wherein the collection unit collects the magnetic resonance data using an ASL (Arterial Spin Labeling) method and changes as the parameter a time from the application of a pre-pulse labeling the fluid to the start of the collection of the magnetic resonance data.

15. The system according to claim 11, wherein the collection unit collects the magnetic resonance data using an FBI (Flesh Blood Imaging) method and changes as the parameter a time from a reference time phase in pulsation of the fluid to the start of the collection of the magnetic resonance data.

16. The system according to claim 11, wherein the collection unit collects the magnetic resonance data using a flow-spoiled FBI method and changes the strength of a diphase pulse as the parameter.
